(19) United States
(12) Patent Application Publication
(10) Pub. No.: US 2005/0158351 A1
(43) Pub. Date: Jul. 21, 2005

(54) Anhydrous skin cleansing and scrubbing composition

(75) Inventors: Nadia Soliman, East Brunswick, NJ (US); Josh Ghaim, Franklin Park, NJ (US)

Correspondence Address: HARNESS, DICKEY & PIERCE, P.L.C., P.O. BOX 8910, RESTON, VA, 20195, US

(73) Assignee: Colgate-Palmolive Company, New York, NY (US)

(21) Appl. No.: 11/010,573

(22) Filed: Dec. 14, 2004

Related U.S. Application Data
(60) Provisional application No. 60/531,559, filed on Dec. 19, 2003.

(51) Int. Cl. A61K 7/00

(52) U.S. Cl. 424/401

(57) ABSTRACT

A substantially anhydrous composition comprising: a) from about 20 to about 90 weight % of at least one water insoluble emollient oil; b) from about 0.5 to about 15 weight % of an ionic surfactant; c) from about 5 to about 40 weight % of an exfoliative and/or polishing material; and d) from about 0.5 to about 10 weight % of at least one oil gelling agent.

This application claims priority to provisional application Ser. No. 60/531,559, filed Dec. 19, 2003, which application is incorporated herein by reference in its entirety for all purposes.

### FIELD OF THE INVENTION

This invention relates to skin cleansing compositions that have an exfoliating action.

### BACKGROUND

Aqueous skin cleansing compositions have been used for centuries. Washing the skin with various surface-active preparations results in the swelling of the horny layers, with water insoluble constituents of dirt being washed off and substances endogenous to the skin being washed out. During this process, naturally occurring skin fats are also removed causing over-drying of the skin, thus making it necessary to replenish the skin with moisturizers such as creams and lotions based on oil-in-water and water-in-oil emulsions.

The most common way of replenishing the skin with moisturizers and conditioners is to use mild cleansers for showering and then applying an aqueous based emulsion such as creams and lotions after showering. These steps are time consuming and not the most effective methods of moisturizing the skin. An effective way to moisturize the skin is to lock-in moisture while wet.

Prior attempts have been made to overcome the foregoing problems of skin cleansers and use of emulsion based creams and lotions by employing high oil content moisturizing body washes based on emulsion technologies or even completely anhydrous skin cleansers. Anhydrous skin cleansers, sometimes called waterless cleansers, typically contain high concentrations of water-insoluble solvents, which makes them generally effective at removing oily undesirable moieties from the skin, but less effective in removing water-soluble undesirables. Further, anhydrous skin cleansers typically are not cosmetically elegant tending to have a heavy, greasy feel making them unappealing to the touch and are not easily removed from the skin. Generally they must be wiped off with toweling, leaving the skin feeling greasy. Alternatively, the skin may be washed off with strong soap, leaving the skin feeling harsh and dry.

Various solutions to these problems have been proposed. A composition within U.S. Pat. No. 6,524,594 describes a foaming system with high gelled oil content. While this form is an improvement over traditional cleansers, the fact that it generates a significant amount of foam, by definition, will remove a significant amount of the desirable moisturizing oils.

U.S. Pat. No. 4,673,526 describes a composition that is limited to cleansing by removal of oily substances on the skin without the addition of fat bodies to the skin. Without being bound by theory, it is believed that this composition works through exfoliation without the presence of foaming surfactants for deep cleansing of the skin, thus leaving the skin feeling greasy and coated.

These and other disadvantages can be overcome by the discovery and use of a new at least substantially anhydrous skin cleansing composition with mild foaming surfactants and exfoliating particulates which not only cleanse the skin but also provide a skin smoothing effect through exfoliation of dead skin cell debris and deposition of significant amounts of emollients to the skin.

The substantially anhydrous skin cleansing composition of the present invention provides effective skin exfoliation and/or a polishing effect while moisturizing and conditioning in a cosmetically pleasing aesthetic vehicle, thus leaving the skin exceptionally soft, smooth and moisturized instantly after showering.

The composition of the present invention has enhanced phase stability, is easily applied to wet skin, and can be effectively removed from the skin with varying amounts of water to leave the skin with perceivable and desirable sensory attribute during and after use.

### SUMMARY OF THE INVENTION

In accordance with the invention, there is provided a substantially anhydrous composition which comprises:
a) at least one water insoluble emollient oil in sufficient quantity to provide emolliency to the skin (for example, in the range of 20-90 weight %);
b) an ionic surfactant in an amount sufficient to provide adequate lather for cleansing (for example, in the range of 0.5-15 weight %);
c) exfoliative and/or polishing material in sufficient quantities to remove cell debris from the skin during application of the composition (for example, in the range of 5-40 weight %); and
d) at least one oil gelling agent in an amount sufficient for enhanced phase stability (for example, in the range of 0.5-10 weight %).

An exemplary aspect of the invention is the application of this composition to wet skin, working the composition into wet skin while cleansing, adding water to the composition on the skin, with continual working of the composition into the skin, if desired, and then removing the composition (and any debris) from the skin as a solution or an aqueous emulsion.

### DETAILED DESCRIPTION OF THE INVENTION

At least one water insoluble or immiscible emollient oil component is present in the composition in the range of about 20-90 weight % of the composition based on the entire weight of the composition. Particular ranges of interest include a minimum of about 25 or 30 weight % of the composition and a maximum amount of about 80, 70 or 60 weight % of the composition. Illustrative examples of the oil(s) include by class followed by particular examples:
a) mineral oils: paraffin oil, petroleum jelly oil;
b) animal oils: Purcellin oil, perhydrosqualene, fish oils and lanolin oil;
c) vegetable oils: sweet almond oil, palm oil, calophyllum oil, avocado oil, olive oil, castor oil, cereal germ oil such as oil of wheat germs, canola oil, sunflower oil, soybean oil and jojoba oil;
d) silicone oils: dimethylpolysiloxane, cyclomethicone such as one or more of D5 and D6 cyclomethicones and mixtures of D5 and D6;
e) organic esters havingl2-36 carbons: butyl myristate, isopropyl myristate, cetyl myristate, isopropyl palmitate, butylstearate, hexadecyl stearate, isopropyl stearate, octyl stearate, isoceryl stearate, decyl oleate, hexyl laurate di-caprylate of propylene glycol, di-isopropyl adipate, myristyl octanoate, and cetyl ricinoleate;
f) organic alcohols (straight chain or branched) having 10-26 carbons: oleic alcohol, linoleic alcohol, linolenic alcohol, isostearyl alcohol, and octyl dodecanol;
g) esters derived from lanolic acid: isopropyl lanolate and isocetyl lanolate;
h) free fatty acids having 12-20 carbons: linoleic, myristic, palmitic, and stearic;
i) acetyl glycerides; and
j) mixtures of any of a)-i).

The ionic surfactant (component b) is also an important portion of the composition. The ability to foam in the presence of the water insoluble (or immiscible) emollient oil (component a) when water is added, is a significant effect of the composition. This foaming ability provides a clean surface to the skin after water is added but still allows the skin to benefit from the emolliency of the oil, employed at a minimum level sufficient to provide creaminess and/or foam to cleanse the skin. Typically, the compositions of the invention comprise from about 0.5-15 weight % of the ionic surfactant with particular values being minimums of about 1, 3 or 5 weight %. In addition to the ionic surfactant, other surfactants may be added so that the total amount of surfactant does not exceed 30 weight %, with particular values being a maximum of about 20 or 25 weight %.

Examples of ionic surfactants include (a) anionic and (b) cationic surfactants.

(a) Anionic surfactants can be exemplified by the alkali metal salts of organic sulfate having in their molecular structure an alkyl radical containing from about 8 to about 22 carbon atoms and a sulfonic acid or sulfuric acid ester radical. Preferred are the sodium, ammonium, potassium or triethanolamine alkyl sulfates; sodium coconut oil fatty acid monoglyceride sulfates and sulfonates; sodium or potassium salts of sulfuric acid esters of the reaction product of 1 mole of higher fatty alcohol (e.g. coconut oil alcohols) and 1 to 12 moles of ethylene oxide; sodium or potassium salts of alkyl phenol ethylene oxide ether sulfate with 1 to 10 units of ethylene oxide per molecule and in which the alkyl radicals contain from 8 to 12 carbon atoms, sodium alkyl glyceryl ether sulfonates; and others known in the art. Examples of anionic surfactants include but are not limited to alkyl sulfates, isethionates (for example, acyl isethionates), sarcosinates (for example, acyl sarcosinates), methyl acyl taurates, glutamates (for example, N-acyl glutamates), alkyl sulfosuccinates, alkyl phosphate esters, ethoxylated alkyl phosphate esters, trideceth sulfates, mixtures of ethoxylated alkyl sulfates and the like. Alkyl chains of these surfactants are from about 8 to 22 carbon atoms, preferably 10 to 18 carbon atoms, more preferably 12 to 16 carbon atoms.

(b) Some examples of cationic surfactants include, but are not limited to, stearalkonium chloride, dodecyltrimethylammonium chloride, stearyldimethylbenzyl ammonium chloride, ditallow(hydrogenated)dimethyl ammonium chloride, and the like. For additional cationic surfactants with long chain alkyl groups see CTFA Cosmetic Ingredient Dictionary, 4^{th} Edition 1991, pages 509-514, incorporated herein by reference.

In addition to the required ionic surfactant component, suitable surfactants that may optionally be additionally included are (c) amphoteric and (d) nonionic surfactants.

(c) Examples of amphoteric surfactants that are suitable for use in the present invention include, but are not limited to, alkyl betaines such as coco-dimethyl carboxymethyl betaine, sulfobetaines such as coco-dimethyl sulfopropyl betaine, stearyl dimethyl sulfopropyl betaine, amidoalkyl betaines, amidoalkyl sultaines, amidosulfobetaines, and the like. Other amphoteric surfactants which can be used in the compositions of the present invention are those which can be broadly described as derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g. carboxy, sulfonated, sulfate, phosphate or phosphonate. Examples of compounds falling under this definition are sodium 3-dodecylaminopropionate, N-alkyltaurines, such as the one prepared by reacting dodecylamine with sodium Isethionate according to the teaching of U.S. Pat. No. 2,438,091.

(d) Nonionic surfactants can be used in the present invention in addition to at least one ionic surfactant. Some examples of nonionic surfactants include, but are not limited to, alkyl polyglucosides wherein the alkyl group has from about 8 to about 16 carbon atoms, and preferably from about 10 to about 16 carbon atoms, e.g. decyl or lauryl glucosides, cocoamidopropyl amine oxides, and the like.

Exfoliative and/or polishing materials (component c) provides a polishing and/or exfoliative action to the skin. Illustrative exemplification of these materials are sugars and hydrophobically or hydrophylically modified polymers such as celluloses.

Examples of exfoliative and/or polishing material include mainly sucrose (cane sugar, beet sugar) and other mono, di or polysaccharides. Monosaccharides are any simple sugars having the formula C₆H₁₂O₆. Disaccharides are ethers formed from two monosaccharides. Polysaccharides are polyethers of monosaccharides which includes starches and celluloses which are hydrophobically or hydrophylically modified. These exfoliative and/or polishing materials should have a mean suitable particle size diameter in the range from 50-1200 microns.

Examples of particular polysaccharides include:
(a) xanthan gums, which are heteropolysaccharides, notably the products known under the commercial names of KELTROL and KELTROL F by the CP KELCO Company, as well as a product known under the commercial name of RHODOPOL 23 and RHODIACARE T sold by RHODIA, INC.;
(b) carboxymethy starches, notably the product sold under the commercial name of PERFECTAMYL GEL 45 and PERFECTAMYL GEL MB by the AVEBE Company;
(c) cellulose ethers such as ethylhydroxyethylcellulose, sold under the commercial name of “BERMOCOLL” by the AKZO NOBEL Company;
(d) hydroxyalkylcelluloses such as hydroxyethylcellulose and hydroxypropylmethylcellulose, sold under the commercial name of CELLOSIZE by AMERCHOL CORPORATION, or under the commercial name of NATROSOL by the HERCULES, INC. or under the commercial name of METHOCEL by the DOW CHEMICAL Company, including METHOCEL E50;
(e) hydroxypropyl starches, notably the products sold under the commercial names of ZEINA B860 by the GRAIN PROCESSING CORPORATION. These polymers desirably have an appropriate granulometry allowing a gentle and non-irritating action;
(f) selected water-soluble materials such as urea and water-soluble urea derivatives such as allantoin can also be used; and
(g) mixtures of the foregoing.

It should be noted that some of these materials are substantially crystalline, but others are not. It should also be noted that the one or more of these water-soluble materials which are employed should:
(1) have sufficient water solubility such that at least about 15 parts by weight of the material will dissolve in 100 parts by weight of water at 20° C., (particularly including materials that have a solubility of at least about 25 or 30 parts by weight) solubility;
(2) be insoluble (solubility to be less than 5 parts by weight based on 100 parts) in the anhydrous skin cleanser composition; and
(3) have a particle size that is sufficiently large to serve as a scrubbing and/or polishing agent against the skin to remove dirt and cellular debris, such as rough dead skin cells, and yet not so large as to scratch or irritate the skin (for example, suitable particle sizes for use with the present invention are those in the range from 50-1200 microns).

Component (c) materials, particularly the sugars, are generally employed at a minimum level sufficient to provide a skin exfoliative, cleansing, skin debris removal effect. Typically, at least about 1, 5, or 10 weight % of component (c) based on the entire weight of the composition is employed, and particularly at least about 20 or 30 weight %. With regard to a maximum amount of component (c), no more than about 50 weight % is used, more particularly a maximum of about 40, 30 or 25 weight %. Mixtures of the various choices listed for component (c) can also be used, for example, mixtures of polymers and mixtures of sugars and polymers can be used.

The oil gelling agent (component d) is the material that provides enhanced phase stabilization to the composition. With its use, phase integrity of the overall composition is substantially improved. When component (d) is absent, the overall moisturization and conditioning efficacy of the product is reduced and the composition has an increased tendency to separate into distinct phases, the lighter oil phase rising to the top with the heavier materials sinking to the bottom. This separation results in an unpleasant appearance of the composition, particularly when it is in a container wherein the composition is “scooped” therefrom as well as presenting a serious potential issue of using a composition richer in one component and/or poorer in another component each time the composition is employed. The use of component (d) also increases the sensory attributes such as moisturization and conditioning as well as improving the phase stabilization as exemplified by the visually homogeneous phase of the composition or at least essentially visually homogeneous phase. With a physically stable phase present, the delivery (storage/packaging) vehicle possibilities are expanded substantially. For example, the composition can be loaded into a container such as a clear tube capable of deformation wherein a uniform or at least substantially uniform composition can be delivered to the skin upon pressure deformation of the container's exterior.

The quantity of oil gelling agent used is that which enhances phase stabilization. Typically, an amount in the range from about 0.5-10 weight % based on the total weight of the composition is suitable. Particular values include a minimum of at least about 1, 1.5 or 2 weight % of the composition can be employed, desirably at least about 3 or 4 weight %. Usually no more than about 7 or 10 weight % of the composition is oil-gelling agent. Mixtures of oil-gelling agents can also be employed.

Examples of oil-gelling agents that can be used include, but are not limited to, silicas, clays and organically modified clays and mixtures thereof. Exemplary oil-gelling silicas preferably include, without being limited thereto, finely divided silicas, magnesium aluminum silicate and the like. Suitable silicas are commercially sold under the trade names Aerosil (Degussa) and Cab-O-Sil (Cabot Corp.). Exemplary oil-gelling clays and organically modified clays preferably include, but are not limited to, bentonites, hectorites, organophilic clays such as Stearalkonium hectorite, Quaternium-18 hectorite, Quaternium-18 bentonite and the like.

With respect to various terms employed in the specification and claims “substantially anhydrous” means less than about 5 weight % water in the composition, preferably less than about 3 weight % water, more preferably less than about 1.5 weight % water, and most preferably 0 to about 1 weight % water. In measuring the water amount, any water of crystallization is not counted in “substantially anhydrous”.

The viscosity of the composition is generally that of a thick liquid or gel but can reach a paste-like consistency. Typically, the viscosity is in the range of about 5,000-8,000,000 centipoises (“cps”). Particular values include a minimum of about 15,000 or 20,000, preferably about 50,000 and a maximum of about 500,000 cps.

Viscosity is measured by standard techniques such as the use of a Brookfield Viscometer. Those skilled in the art will use the appropriate spindle and speed combination to cover the range of viscosity to be measured. For less viscous samples, Brookfield spindle #5, at 20 rpm and 25° C. is suitable. At high viscosities, a helipath attachment is used with; for example, spindle T-D at 10 rpm and 25° C. For example, a range of viscosity of about 500,000 to 2,000,000 cps is measured with a Brookfield viscometer using a helipath attachment with a T-D spindle at 10 rpm and about 25° C.

The compositions of the invention can optionally also contain dyes, perfumes, antioxidants, antimicrobials, sensory signal agents (cooling or warming), skin protective agents such as UV absorbers, and other auxiliary materials that are conventionally used personal care/cosmetic compositions.

Examples of suitable antioxidants include, but are not limited to, vitamins E, A, C and their ester forms, niacin, choline, carnitine, panthenol, biotin and mixtures thereof.

Examples of suitable antimicrobials include, but are not limited to, trichlorocarbanilide, triclosan, benzalkonium chloride, phenol and mixtures thereof.

Examples of suitable sensory signals include, but are not limited to, magnesium chloride, ammonium chloride, magnesium sulfate and the like, that produce a warming or cooling sensation to the touch during use in the presence of water due to the heat of solution resulting in endothermic or exothermic reaction.

Examples of suitable skin protecting sunscreen actives include, but are not limited to, octyl methoxycinnamate, benzophenones, octocrylene, titanium dioxide, zinc oxide and mixtures thereof.

The amount of optional agents to be combined with the composition (of the present invention) may vary depending upon, for example, the benefit or visual aesthetics desired and the sensitivity of user to the optional agent. Preferably the optional ingredient is present in the composition (of the present invention) in an amount, from 0.01 weight % to 15 weight % and more preferably from about 0.01 weight % to 10 weight %, and even more preferably from 0.01 weight % to 5 weight %.

The anhydrous skin compositions of this invention can be prepared by mixing together the emollient water-immiscible oil ingredients and oil-gelling agent ingredients at room temperature with sufficient mixing agitation to dissolve the oil-gelling agent, with homogenization if needed. The surfactants are then added with mixing agitation and homogenization if necessary, until a homogenous mixture is formed. The exfoliating/polishing ingredient and fragrance are then added with continuous agitation to form the finished product. Other benefit and visual aesthetics ingredients can be added at the end of the process, if desired. Those skilled in the art will understand that the order of incorporation of ingredients and temperatures employed may vary with the type of ingredient and the manner of dissolution recommended by the supplier of the material.

A preferred embodiment for using substantially anhydrous skin compositions formulated according to the invention comprises the following steps:
(a) applying the anhydrous skin cleanser to wet skin, preferably by manually rubbing the applied amount over the skin to generate foam and thoroughly coat the skin (rubbing action preferably is a gentle rubbing or massaging for a period that is appropriate for normal cleansing, to promote the removal of oily or greasy, water-insoluble soils and skin cell residues);
(b) contacting the skin cleanser coated skin with an amount of water sufficient to moisten the coated skin;
(c) continuously rubbing and massaging the so-moistened skin until the abrasive ingredient particles substantially dissolve;
(d) removing the resultant emulsion from the skin, preferably by rinsing it off with additional water; and
(e) drying the skin.
   Note that effective skin emolliency and removal of the composition can both be accomplished by adding water of small, medium or large quantities. The skin can be dried by gentle blotting or toweling the skin as well as by letting the skin air dry.

Anhydrous skin formulations prepared with the component and ranges disclosed possess a surprising combination of beneficial effects when used on soiled skin. The term “soil” as applied to skin includes soils from natural sources, such as cellular debris present on the skin, and soils from external sources topically applied to the skin, such as from makeup, cosmetics, industrial greases, and environmental dirt. The anhydrous skin compositions can thoroughly cleanse the skin of makeup and other water-immiscible cosmetic residues. They effectively remove various types of oily and greasy soils from the skin surface and difficult to solubilize residues. The anhydrous skin compositions can remove rough and dry skin cells. Moreover, the anhydrous skin compositions can smooth and polish the skin surface and leave the skin soft and moisturized. The following examples further illustrate the anhydrous skin compositions of this invention with specific embodiments, ingredients and methods but are not intended to be limiting.

### EXAMPLES

The following Examples are offered as illustrative of the invention and are not to be construed as limitations thereon. In the Examples and elsewhere in the description of the invention, chemical symbols and terminology have their usual and customary meanings. In the Examples as elsewhere in this application values for n, m, etc. in formulas, molecular weights and degree of ethoxylation or propoxylation are averages. Temperatures are in degrees C. unless otherwise indicated. The amounts of the components are in weight percents based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7^{th} ed. 1997).

### Example 1

Using the following ingredients, a composition can be made by the following method. The composition in Example 1 can be prepared by mixing together the vegetable oil silica at room temperature with sufficient mixing agitation to dissolve the silica, with homogenization if needed. The sodium cocoyl isethionate surfactant and dimethicone are then added with mixing agitation and homogenization if necessary, until a homogenous mixture is formed. The granulated sugar and fragrance are then added with continuous agitation to form the finished product. Other benefit and visual aesthetics ingredients can be added at the end of the process, if desired. Suitable compositions can be made with the following amounts of ingredients:
20-90 weight % oil, for example a vegetable oil like sunflower oil (preferably 30-85 weight % and, more preferably, 50-75 weight %);
0.5-10 weight % silica (preferably 1-6 weight % and, more preferably, 2-5 weight %)
0.5-15 weight % sodium cocoyl isethionate (preferably 1-6% and, more preferably, 2-5 weight %);
0-5 weight % silicone oil e.g. dimethylpolysiloxane (preferably 0.1-4 weight % and, more preferably, 2-3 weight %); and
10-40 weight % sucrose (preferably 12-30 weight % and, more preferably, 15-25 weight %).

### Example 2-8

The method of Example 1 may be repeated with the ingredients listed in TABLE A.

TABLE A

| | Ex. 2 | Ex. 3 | Ex. 4 | Ex. 5 | Ex. 6 | Ex. 7 | Ex. 8 |
|---|---|---|---|---|---|---|---|
| Components | Wt % | Wt % | Wt % | Wt % | Wt % | Wt % | Wt % |
| Sunflower Oil | 56.0 | 67.8 | 57.0 | 59.0 | 56.0 | 49.0 | 67.8 |
| Mineral Oil | — | — | — | 10.0 | — | — | — |
| Fumed Silica | 5.0 | 3.0 | 5.0 | 3.0 | 3.0 | 5.0 | 4.0 |
| Sodium Cocoyl Isethionate | 3.0 | 3.0 | 2.0 | 3.0 | 3.0 | — | 5.0 |
| Cocoamidopropyl Betaine | 2.0 | 3.0 | 5.0 | 2.0 | 2.0 | 10.0 | — |
| Laureth-4 | — | — | — | — | — | 5.0 | — |
| Dimethyl Polysiloxane (60K cst) | 2.0 | 2.0 | — | 2.0 | 2.0 | — | 2.0 |
| Sucrose Granulated | 30.0 | 20.0 | 30.0 | 20.0 | — | 30.0 | 20.0 |
| Sodium Chloride Granules | — | — | — | — | 30.0 | — | — |
| Fragrance | 1.0 | 1.20 | 1.0 | 1.0 | 1.0 | — | — |
| Benefits Agents (vitamin E Acetate) | 1.0 | — | — | — | — | 1.0 | 1.2 |
| Totals | 100% | 100% | 100% | 100% | 100% | 100% | 100% |
| Viscosity (×10000 cps) | 6,000* | 25** | 190** | 50** | 25** | >6,000* | 100** |

*Viscosity was measured using T-E at 0.5 rpm at 25 deg. C.
**Viscosity was measured using T-D at 10 rpm at 25 deg. C.

Samples from Examples 2-8 were placed in 120 ml (4 ounce) glass containers and allowed to age at various temperatures as described below. Appearance of phase separation was examined by visual inspection. Amount of separation was estimated by dividing the volume of separated liquid by the total volume in the sample container.

### Example 2

No visual phase separation occurred at 43 degrees C. after 8 weeks of aging with acceptable foam and creaminess for cleansing the skin.

### Example 3

Approximately 1% phase separation was observed at 43 degrees C. after 8 weeks of aging with acceptable foam and creaminess for cleansing the skin.

### Example 4

No visual separation occurred at 43 degrees C. after 8 weeks of aging with acceptable foam but reduced creaminess and spreadability issues during the cleansing of the skin.

### Example 5

Approximately 5% phase separation was observed at room temperature with significantly depressed foam and creaminess during cleansing of the skin.

### Example 6

Approximately 1% phase separation was observed at 43 degrees C. after 12 weeks of aging with no foam due to the presence of salt but more of a creamy texture during cleansing of the skin.

### Example 7

No visual separation occurred at 43 degrees C. after 8 weeks of aging with significantly higher foam but reduced creaminess and spreadability issues during the cleansing of the skin.

### Example 8

No visual separation occurred at 43 degrees C. after 8 weeks of aging with significantly higher foam and foam creaminess. Product spreadability during the cleansing of the skin was optimal.

All citations to books, magazines, journal articles, patents, or any other publications, etc., recited in this application are expressly incorporated herein by reference in their entirety for all purposes.

1. A substantially anhydrous composition comprising:
a) from about 20 to about 90 weight % of at least one water insoluble emollient oil in sufficient quantity to provide emolliency to the skin;
b) from about 0.5 to about 15 weight % of an ionic surfactant to provide sufficient lather for cleansing;
c) from about 5 to about 40 weight % of an exfoliative and/or polishing material in sufficient quantities to remove cell debris from the skin during application of the composition; and
d) from about 0.5 to about 10 weight % of at least one oil gelling agent in sufficient quantity for enhanced phase stability.

2. A composition in accordance with claim 1, wherein the water insoluble emollient oil is selected from the group consisting of:
a) mineral oils;
b) animal oils;
c) vegetable oils;
d) silicone oils;
e) organic esters having 12-36 carbons;
f) organic alcohols having 10-26 carbons;
g) esters derived from lanolic acid;
h) free fatty acids having 12-20 carbons;
i) acetyl glycerides; and
j) mixtures of any of a)-i).

3. A composition in accordance with claim 2, wherein the water insoluble emollient oil is selected from the group consisting of:
a) mineral oils selected from the group consisting of paraffin oil and petroleum jelly oil;
b) animal oils selected from the group consisting of Purcellin oil, perhydrosqualene, fish oils and lanolin oil;
c) vegetable oils selected from the group consisting of sweet almond oil, palm oil, calophyllum oil, avocado oil, olive oil, castor oil, wheat germ oil, canola oil, sunflower oil, soybean oil and jojoba oil;
d) silicone oils selected from the group consisting of dimethylpolysiloxane, D5 cyclomethicone, D6 cyclomethicone, and mixtures of D5 and D6 cyclomethicones;
e) organic esters having 12-36 carbons;
f) straight and branched chain organic alcohols having 10-26 carbons;
g) esters derived from lanolic acid and selected from the group consisting of isopropyl lanolate and isocetyl lanolate;
h) free fatty acids having 12-20 carbons;
i) acetyl glycerides; and
j) mixtures of any of a)-i).

4. A composition in accordance with claim 3, wherein the water insoluble emollient oil is selected from the group consisting of:
(a) organic esters selected from the group consisting of myristate, isopropyl myristate, cetyl myristate, isopropyl palmitate, butylstearate, hexadecyl stearate, isopropyl stearate, octyl stearate, isoceryl stearate, decyl oleate, hexyl laurate di-caprylate of propylene glycol, di-isopropyl adipate, myristyl octanoate, and cetyl ricinoleate;
(b) organic alcohols selected from the group consisting of oleic alcohol, linoleic alcohol, linolenic alcohol, isostearyl alcohol and octyl dodecanol; and
(c) free fatty acids selected from the group consisting of linoleic, myristic, palmitic, and stearic.

5. A composition in accordance with claim 1 wherein the ionic surfactant comprises an anionic surfactant selected from the group consisting of alkali metal salts of organic sulfate having an alkyl radical containing from about 8 to about 22 carbon atoms and a sulfonic acid or sulfuric acid ester radical.

6. A composition in accordance with claim 5 wherein the anionic surfactant is selected from the group consisting of sodium, ammonium, potassium and triethanolamine alkyl sulfates; sodium coconut oil fatty acid monoglyceride sulfates and sulfonates; sodium and potassium salts of sulfuric acid esters of the reaction product of 1 mole of higher fatty alcohol and 1 to 12 moles of ethylene oxide; sodium and potassium salts of alkyl phenol ethylene oxide ether sulfate with 1 to 10 units of ethylene oxide per molecule and in which the alkyl radicals contain from 8 to 12 carbon atoms, sodium alkyl glyceryl ether sulfonates.

7. A composition in accordance with claim 5 wherein the anionic surfactant is selected from the group consisting of alkyl sulfates, isethionates, sarcosinates, methyl acyl taurates, glutamates, alkyl sulfosuccinates, alkyl phosphate esters, ethoxylated alkyl phosphate esters, trideceth sulfates, mixtures of ethoxylated alkyl sulfates, all of which have alkyl chains of from about 8 to 22 carbon atoms.

8. A composition in accordance with claim 1 wherein the ionic surfactant comprises a cationic surfactant.

9. A composition in accordance with claim 1 wherein the ionic surfactant comprises a member selected from the group consisting of stearalkonium chloride, dodecyltrimethylammonium chloride, stearyldimethylbenzyl ammonium chloride, and ditallow(hydrogenated)dimethyl ammonium chloride.
